(19) United States
(12) Patent Application Publication
(10) Pub. No.: US 2020/0197323 A1
(43) Pub. Date: Jun. 25, 2020

(54) METHODS FOR SUPPRESSING ACCUMULATION OF REFLUX-INDUCED PROTEIN ADDUCTS

(71) Applicant: Vanderbilt University, Nashville, TN (US)

(72) Inventors: Alexander Zaika, Nashville, TN (US); Sergey I. Dikalov, Nashville, TN (US)

(21) Appl. No.: 16/724,142

(22) Filed: Dec. 20, 2019

Related U.S. Application Data
(60) Provisional application No. 62/783,998, filed on Dec. 21, 2018.

(51) Int. Cl. A61K 31/06; A61P 1/04

(52) U.S. Cl. CPC A61K 31/06; A61P 1/04

(57) ABSTRACT

Compounds and methods for suppressing or ameliorating accumulation of reflux-induced protein adducts to a subject in need thereof, comprising administering an effective amount of a compound of the following formula:

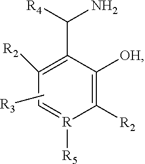

wherein the variables are defined herein.

### PRIOR APPLICATIONS

This application claims benefit to U.S. Patent Application No. 62/783,998, filed Dec. 21, 2018, the contents of which are incorporated herein by reference.

### GOVERNMENT INTEREST

This invention was made with government support under grant numbers RO1 206564 and RO1 138833 awarded by the National Cancer Institute. The government has certain rights in the invention.

### TECHNICAL FIELD

One embodiment of the present invention relates to articles and methods for suppressing accumulation of reflux-induced protein adducts. In particular, the presently-disclosed subject matter relates to articles and methods for suppressing isolevuglandins (isoLG) protein adducts associated with gastroesophageal reflux.

### BACKGROUND AND SUMMARY

Anatomically, the upper gastrointestinal tract consists of the mouth, a portion of the throat, the esophagus, the stomach and the duodenum, the uppermost part of the small intestine.

The esophagus carries food, liquids, and saliva from the mouth to the stomach by coordinated contractions of its muscular lining. The muscular layers of the esophagus are normally pinched together at both the upper and lower ends by muscles called sphincters. When a person swallows, the sphincters relax automatically to allow food or drink to pass from the mouth into the stomach. The muscles then close rapidly to prevent the swallowed food or drink from leaking out of the stomach back into the esophagus or into the mouth. When people belch to release swallowed air or gas from carbonated beverages, the sphincters relax and small amounts of food or drink may come back up briefly; this condition is called reflux. The esophagus quickly squeezes the material back into the stomach. This amount of reflux and the reaction to it by the esophagus are considered normal.

Bile reflux occurs when bile and other digestive fluids flow upward (refluxes) from the small intestine into the stomach and then into the esophagus. Bile reflux may accompanies acid reflux, and together they may cause inflammation of the esophageal lining and increased risk of esophageal cancer.

Disorders and/or symptoms that are believed to be associated with bile reflux, either alone or in combination with acid reflux, include, for instance, heartburn, indigestion, dyspepsia, erosive esophagitis, peptic ulcer, gastric ulcer, esophageal ulcers, esophagitis, laryngitis, pharyngitis, coarse or hoarse voice, and GERD-related pulmonary dysfunction such as coughing and/or asthma. Further complications that are believed to occur as a result of chronic bile reflux are, for instance, gastroesophageal reflux disease (GERD); Barrett's esophagus; esophageal cancer (e.g., adenocarcinoma) and gastritis.

GERD is a generic term encompassing diseases with various digestive symptoms such as pyrosis, acid regurgitation, obstructed admiration, aphagia, pectoralgia, permeating feeling and the like sensibility caused by reflux in the esophagus and stagnation of gastric contents, duodenal juice, pancreatic juice and the like. The term covers both reflux esophagitis in which erosion and ulcers are endoscopically observed, and esophageal regurgitation-type non-ulcer dyspepsia (NUD) in which no abnormality is endoscopically observed. GERD occurs when the LES does not close properly and stomach contents leak back, or reflux, into the esophagus.

Esophageal adenocarcinoma (EAC) is one of the fastest rising malignancies in western countries, including the Unites States. Epidemiological studies identified GERD to be the strongest known risk factor for EAC. During an episode of esophageal reflux, epithelial cells are exposed to acidic gastric juice frequently mixed with duodenal bile that cause cell and tissue injury. It also results in induction of DNA damage that can further exacerbate pathological alterations. Among proteins, which are frequently inactivated during development of esophageal tumors, is p53 tumor suppressor. The most common mechanism of p53 inactivation is an acquisition of mutations in the p53 gene (TP53). In fact, TP53 gene is the most mutated gene in EAC. It has also been reported that activity of wild-type p53 protein is directly affected by reflux components. Deoxycholic acid, a bile component, decreases levels of p53 protein and induces its proteasomal degradation. Inhibition of p53 impedes many critical cellular processes, including response to DNA damage and cell cycle control. p53 activity is particularly important in cells exposed to genotoxic agents, such as acidic bile salts, which induce strong DNA damage in esophageal cells. In these conditions, inhibition of p53 may increase mutation rate, exacerbate genomic instability and facilitate tumorigenesis.

The present inventors show that aberrant induction of reactive oxygen species (ROS) by reflux significantly contribute to esophageal tumorigenesis. Excessive production of ROS interfere with functions of various cellular macromolecules, including proteins and DNA. The role of lipid peroxidation is less understood and its role in carcinogenesis has not been elucidated. Among molecules formed as a result of lipid peroxidation are isolevuglandins (isoketals or γ-ketoaldehydes). Isolevuglandins (isoLG) are highly reactive with free amines on lysine residues forming LG-lysine lactam protein adducts, protein-protein and protein-DNA crosslinks that may interfere with normal function of DNA and cellular proteins. The accumulation of modified proteins is seen in pathological conditions, including lung fibrosis and hypertension.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-C show graphs and images illustrating induction of isoLG protein adducts through treatment of esophageal cells with acidic bile salts (BA/A). (A) EPC-2 and TE-7 cells were treated with BA/A (100 μM, pH 4.0) for 15 min and 30 min, respectively and analyzed for isoLG protein adducts at the indicated time points. Adduct levels were determined by densitometry. Treatment with BA/A led to significant accumulation of isoLG adducts in TE-7 (p<0.01; n=3) and EPC-2 (p<0.05; n=3) cells. (B-C) Representative images of isoLGpositive cells. Analyses were performed using immunofluorescence with D11 antibody after treatment with BA/A for 18 hours. Cell nuclei were stained with DAPI. Significant increase in isoLG-positivity was found in treated EPC-2 (B) and TE-7 (C) cells (***p<0.001; n=2).

FIGS. 2A-B show representative chromatograms of lysyl-LG lactam adducts obtained by LC/ESI/MS/MS. (A) Quantification was based on specific transitions from the molecular ion at m/z=479.2 to the specific fragment at m/z=332.1 (m/z=485.2 to 332.1 for the isotopically labeled internal standard). The areas under the curves for each peak are integrated and quantitation is calculated by isotopic dilution. The traces presented are representative for each experimental condition. (B) Levels of LG-lysine lactam adducts were found to be higher in BA/A-treated samples compared to control and treated with isoLG scavenger 2-HOBA.

FIGS. 3A-B show graphs and images illustrating analyses of isoLG adducts in vivo. (A) Representative images of immunohistochemical staining with isoLG-specific D11 antibody in esophageal tissues collected from mice, in which reflux was induced by esophagojejunostomy, and control animals with sham surgery. Surgical induction of reflux significantly increased levels of isoLG adducts in esophageal tissues. Treatment of surgical mice with 2-HOBA inhibited accumulation of isoLG adducts in vivo (p<0.05; n=20). (B) Representative images of immunohistochemical staining of esophageal biopsies collected from patients with and without GERD. Levels of isoLG adducts were assessed using immunohistochemistry with D11 antibody. Some GERD patients showed accumulation of isoLG adducts in esophageal tissues (p=0.07; compared to healthy adults).

FIGS. 4A-C show graphs illustrating how exposure of esophageal cells to acidic bile salts leads to formation of p53 protein adducts and p53 protein precipitation. (A-B) EPC-2 (A) and HET1A (B) cells were treated with BA/A (100M, pH 4.0) for 15 min and 30 min, respectively and analyzed for p53 protein in soluble and insoluble cellular fractions at the indicated time. Western blot membranes were quantitated by densitometry. p53 protein was accumulated in insoluble fraction after exposure to BA/A. Treatment with 2-HOBA increased levels of p53 protein in soluble fraction in both EPC-2 and HET1A cells (* p<0.05, **p<0.01 and ***p<0.001; ns=Not significant). (C) p53 protein was immunoprecipitated with p53(DO-1) antibody and analyzed for isoLG adducts using Western blotting with D11 antibody. IsoLG adducts were quantitated by densitometry. Exposure to BA/A significantly increased levels of p53 protein adducts (* p<0.05; n=3)

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including all definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “gastric fluid,” “gastric refluxate” and “gastric juice” are used interchangeably throughout the disclosure and refer to the endogenous fluid medium of the stomach, including water and secretions.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As can be seen herein, there is overlap in the definition of treating and preventing.

As used herein, the term “diagnosed” means having been subjected to a physical examination by a person of skill, for example, a physician, and found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. For example, a subject can be identified as having a need for treatment of a disorder (e.g., a disorder related to GERD) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “scavenger” or “scavenging” refers to a chemical substance that can be administered in order to remove or inactivate impurities or unwanted reaction products. For example, the isoketals irreversibly adduct specifically to lysine residues on proteins. The isoketal scavengers of the present invention react with isoketals before they adduct to the lysine residues. Accordingly, the compounds of the present invention “scavenge” isoketals, thereby preventing them from adducting to proteins.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “hydroxyl” as used herein is represented by a formula —OH.

The term “nitro” as used herein is represented by a formula —NO₂.

The term “pharmaceutically acceptable” describes a material that is not biologically or otherwise undesirable, i.e., without causing an unacceptable level of undesirable biological effects or interacting in a deleterious manner.

Embodiments of the present invention include compounds of the following formula, and their use as agents in a method for suppressing or ameliorating accumulation of reflux-induced protein adducts in a subject in need thereof:

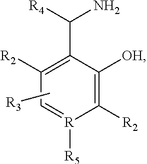

wherein:

R is C;

R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;

R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;

R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;

R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N;

and stereoisomers and analogs thereof; and pharmaceutically acceptable salts thereof.

In other embodiments, the reflux-induced protein adducts comprise isolevyglandins (isoLG) adducts of proteins.

In other embodiments, the subject has GERD or is at risk of having GERD.

Another embodiment of the present invention includes compounds of the following formula, and their use in methods for treating, preventing, or ameliorating bile damage to the esophagus, for subject in need thereof:

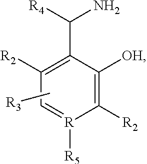

wherein:

R is C;

R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;

R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;

R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;

R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

In other embodiments, the subject has GERD or is at risk of having GERD.

Yet another embodiment of the present invention includes compounds of the following formula, and their use in ameliorating the risk of esophageal adenocarcinoma for a subject in need thereof:

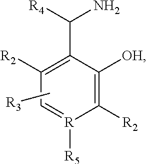

wherein:

R is C;

R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;

R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;

R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;

R₅ is a bond, H, hydroxy, halogen, nitro, CF₃, C₁₋₆ alkyl, C₁₄ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

In other embodiments, the subject has GERD or is at risk of having GERD.

In other embodiments, the esophageal adenocarcinoma is reflux-induced.

Another embodiment of the present invention includes compounds of the following formula, and their use in reducing or ameliorating reactive oxygen species produced by reflux for a subject in need thereof:

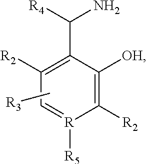

wherein:

R is C;

R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;

R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;

R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;

R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

In other embodiments, the subject has GERD or is at risk of having GERD.

Another embodiment of the present invention is a method for treating, preventing, or ameliorating esophageal damage caused by reflux, comprising the step of co-administering to the subject at least one compound of the following formula:

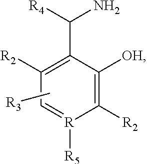

wherein:

R is C;

R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;

R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;

R₄ is H, hydroxy, halogen, nitro, CF₃, C₁₋₆ alkyl, C₁₄ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;

R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; with a drug having a known side effect of treating, preventing, or ameliorating reflux.

In certain embodiments, the compound of the present invention may be selected from the compounds disclosed herein. In a preferred embodiment, the compound used in the methods disclosed herein may be salicylamine (2-HOBA, 2-hydroxybenzylamine), methyl-2-hydroxybenzylamine, ethyl-2-hydroxybenzylamine.

Examples of compounds that may be used with the methods disclosed herein include, but are not limited to, compounds selected from the formula:

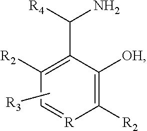

wherein:

R is CH, C—R₃, C—CH₃, or C—CH—CH₃;

R₂ is independently H, substituted or unsubstituted alkyl;

R₃ is independently H, halogen, alkoxy, hydroxyl, nitro;

R₄ is H, substituted or unsubstituted alkyl, carboxyl; and pharmaceutically acceptable salts thereof.

In a preferred embodiment, the compound is salicylamine (2-hydroxybenzylamine or 2-HOBA).

In other embodiments, the compound may be chosen from:

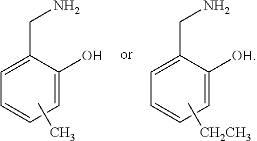

In other embodiments, the compound may be chosen from:

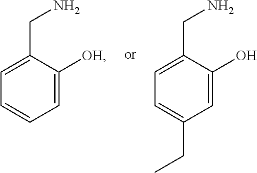

or a pharmaceutically acceptable salt thereof.

The compounds or analogs may also be chosen from:

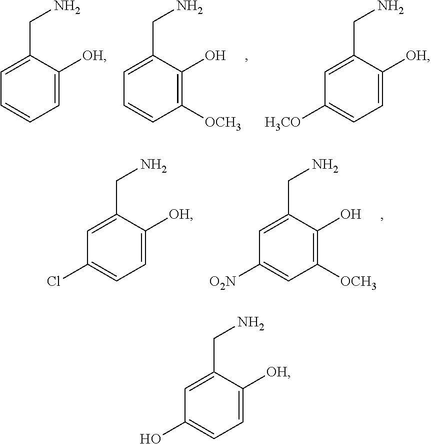

or a pharmaceutically acceptable salt thereof.

The compounds may also be chosen from:

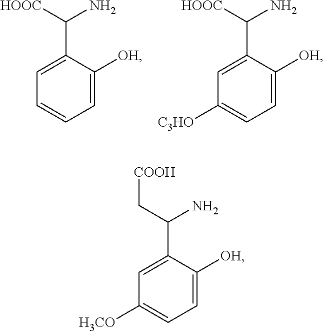

or a pharmaceutically acceptable salt thereof.

Provided herein are articles and methods for suppressing accumulation of reflux-induced protein adducts. In some embodiments, the method includes administering a lipid peroxidation product scavenger to a subject in need thereof. The lipid peroxidation product scavenger may be any scavenger of products of oxidative modification of lipids. For example, in one embodiment, the lipid peroxidation product includes isolevuglandins (isoLG), which are produced when esophageal cells are exposed to refluxate. In another embodiment, the lipid peroxidation product scavenger includes 2-hydroxybenzylamine (2-HOBA). In a further embodiment, the administration of 2-HOBA reduces or eliminates accumulation of isoLG adducts.

In some embodiments, the lipid peroxidation products are highly reactive, and form protein adducts. For example, in one embodiment, isoLG forms protein adducts with p53 tumor suppressor, which is known to play a critical role in prevention of esophageal tumorigenesis. In another embodiment, administration of 2-HOBA reduced and/or inhibited formation of p53 protein adducts by isoLG. Accordingly, in some embodiments, administration of the lipid peroxidation product scavenger to a subject in need thereof prevents, reduces, and/or treats tumorigeneses. Such tumorigeneses includes, but is not limited to, esophageal tumorigeneses, such as that caused by GERD.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to a subject so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

There are numerous medications available that treat reflux, which when used herein includes heartburn and indigestion. Presently, the main therapies employed in the treatment of GERD and upper GI tract disorders include agents for reducing the stomach acidity, for example by using the histamine Hz-receptor antagonists or proton pump inhibitors (PPIs). H2 blockers are drugs that inhibit the production of acid in the stomach. Exemplary histamine Hz-receptor antagonists include, for example, cimetidine (as sold under the brand-name TAGAMET HB®), famotidine (as sold under the brand-name PEPCID AC®), nizatidine (as sold under the brand-name AXID AR®), and ranitidine (as sold under the brand-name ZANTAC 75®). Both types of medication are effective in treating heartburn caused by acid reflux and usually eliminate symptoms within a short period of time.

PPIs act by inhibiting the parietal cell H+/K+ATPase proton pumps responsible for acid secretion from these cells. PPIs, such as omeprazole and its pharmaceutically acceptable salts are disclosed, for example, in EP 05129, EP 124495 and U.S. Pat. No. 4,255,431.

The gastric-retentive, sustained-release oral dosage forms comprising at least one bile acid sequestrant described herein can be used in combination therapy with one or more additional therapeutic agents. For combination treatment with more than one active agent, where the active agents may be in separate dosage forms, the active agents may be administered separately or in conjunction. In addition, the administration of one agent may be prior to, concurrent to, or subsequent to the administration of the other agent.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject. Thus, the methods can include administering simultaneously, separately, or sequentially, a therapeutically effective amount of one or more proton pump inhibitors.

In other embodiments, the methods can include administering simultaneously, separately or sequentially, a therapeutically effective amount of one or more acid pump antagonists.

In other embodiments, the methods can include administering simultaneously, separately, or sequentially one or more agents chosen from an antacid, a histamine H2-receptor antagonist, a γ-aminobutyric acid-3 (GABA-B) agonist, a prodrug of a GABA-B agonist, and a protease inhibitor.

When co-administered with other agents, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

Gastroesophageal reflux disease (GERD) is a digestive disorder characterized by repeated damage of esophageal tissues by acidic gastric refluxate. Epidemiological studies found that GERD is one of the strongest risk factors for esophageal adenocarcinoma, although the specific mechanism(s) causing tumor development remain poorly understood. This Example investigates how esophageal reflux and reflux-associated reactive oxygen species (ROS) affect intracellular proteins. It was found that exposure of esophageal cells to the refluxate leads to production of highly reactive isolevuglandins (isoLG), products of oxidative modification of lipids, that form protein adducts. Increased levels of isoLG adducts of proteins were found in mice in which reflux was induced by surgical procedure and human GERD patients. One of the proteins found to be adducted is p53 tumor suppressor, which is known to play a critical role in prevention of esophageal tumorigenesis. Exposure of esophageal cells to reflux components caused formation of p53 protein adducts and precipitation of p53 protein. IsoLG scavenger, 2-hydroxybenzylamine (2-HOBA), significantly inhibited formation of p53 protein adducts. 2-HOBA also efficiently decreased isoLG protein adducts in mice in vivo. Combined, these studies suggest that modification of p53 and other proteins by reactive products of lipid oxidation may strongly contribute to esophageal tumorigeneses caused by GERD.

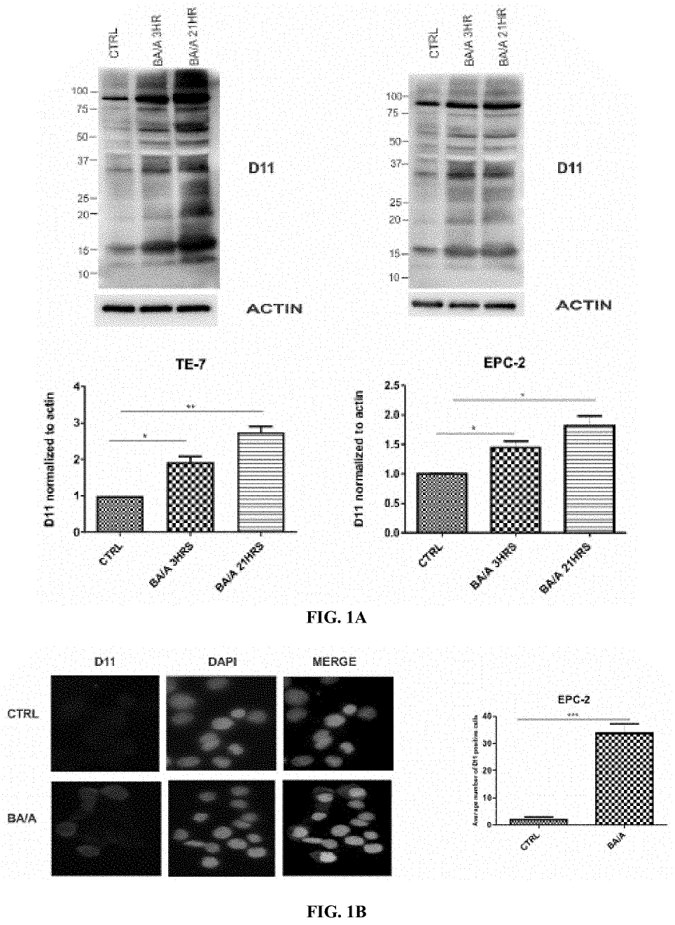

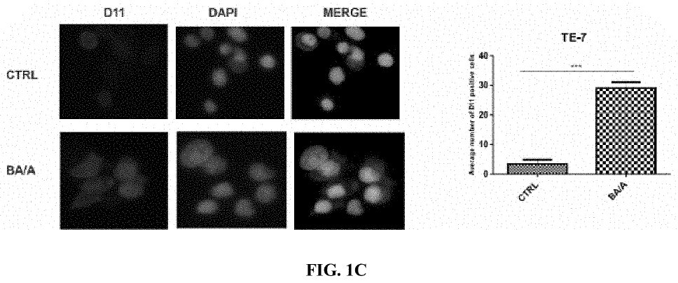

RESULTS AND DISCUSSION: Esophageal reflux creates the environment where esophageal cells are exposed to high concentration of bile components at low pH. Currently little is known about cellular alterations induced by reflux and how these alterations accelerate esophageal tumor development. In order to understand the effects of reflux and reflux-associated ROS, the present inventors explored the formation of isoLG protein adducts. To recapitulate a typical episode of reflux, TE-7 esophageal cells were shortly exposed to acidic growth medium (pH 4.0), supplemented with 100 μM bile salts cocktail (BA/A). The composition, total bile salts concentration and pH were selected based on previous measurements in GERD patients. Treated cells were then collected and analyzed for the formation of isoLG by Western blotting with D11 single-chain antibody that specifically recognizes isoLG protein adducts independently of amino acid sequences of proteins. The present inventors found that treatment of esophageal cells with acidic bile salts (BA/A) leads to significant accumulation of isoLG protein adducts compared to untreated control (FIG. 1A; left panel). Notably, multiple proteins were adducted after BA/A treatment indicated by the strong increase in intensities of multiple protein bands. Given that isoLGs can produce intra-molecular crosslinks, it is also possible that crosslinked proteins may contribute to accumulation of high molecular weight protein products. These experiments were repeated using non-transformed EPC-2 esophageal cells isolated from the normal human esophagus. Similar to TE-7 cells, exposure of EPC-2 cells to BA/A led to significant accumulation of isoLG protein adducts (FIG. 1A; right panel).

To confirm findings, isoLG protein adducts were analyzed by immunofluorescence. EPC-2 and TE-7 cells were treated with BA/A as described above and analyzed for isoLG formation using D11 antibody. Similar to Western blot analyses, BA/A led to significant increase in D11 positivity in both EPC-2 and TE-7 cells (FIG. 1B). Treated cells showed both cytoplasmic and nuclear staining.

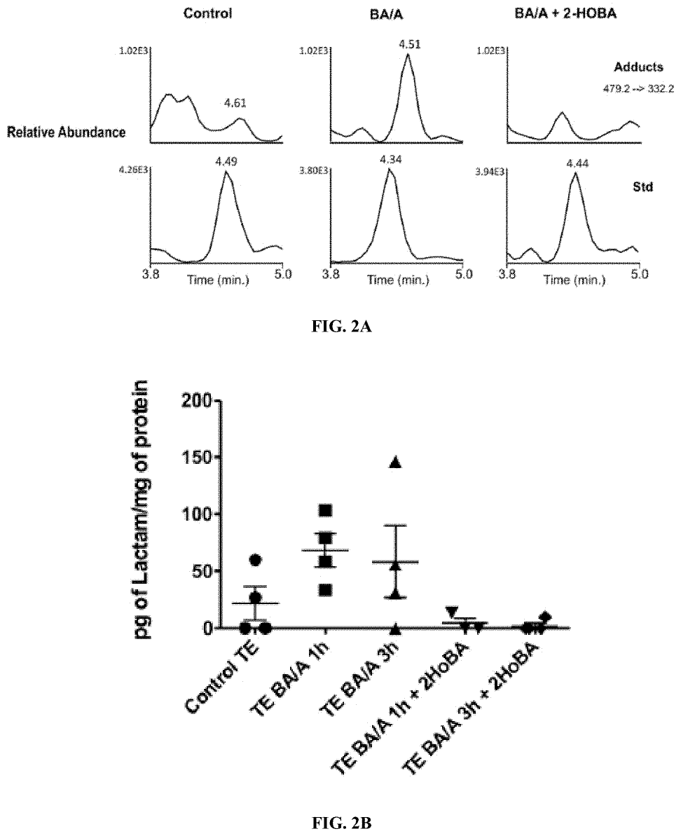

Formation of LG-lysine adducts was further verified by quantification of the LG-lysyl lactam adducts using liquid chromatography-electrospray ionization-tandem mass spectrometry (LC/ESI/MS/MS) in TE-7 cells treated with BA/A. Our studies confirmed that levels of LG-lysine lactam adduct are higher in BA/A treated than control samples. Our quantification analyses were based on specific transitions from the molecular ion at m/z=479.2 to the specific fragment at m/z=332.1. The elution of the two fragment ions found in BA/A treated samples was similar to the [13C6]lysine-lactam internal standard (FIG. 2A). As an additional control, TE-7 cells were treated with BA/A as above but in the presence of known isoLG scavenger 2-hydroxybenzylamine (2HOBA) and analyzed by mass spectrometry. The present inventors found that 2HOBA inhibited formation of isoLG protein adducts (FIG. 2).

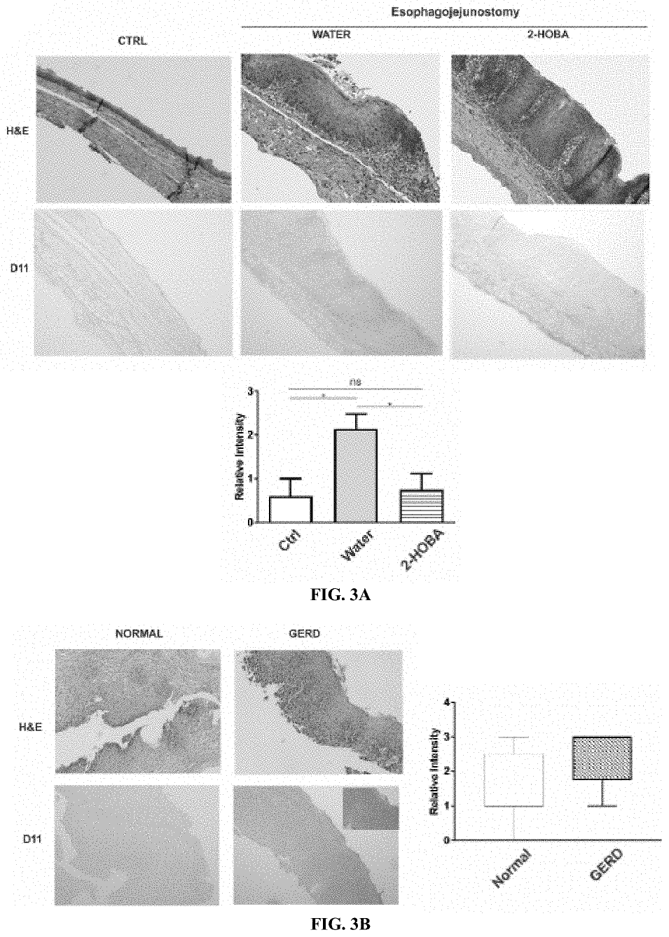

To investigate the formation of isoLG protein adducts in the esophagus, The present inventors used a surgical model of esophageal reflux injury. To generate animals with reflux, a section of the mouse jejunum was transected and then anastomosed to the esophagus resulting in an increased reflux. The present inventors have successfully used this animal model to demonstrate reflux-induced DNA damage. Using immunohistochemistry with D11 antibody, the present inventors compared levels of isoLG protein adducts in esophageal epithelia of animals with reflux induced by surgery and control animals with sham surgery. Levels of isoLG adducts were also analyzed in surgical animals treated with isoLG scavenger 2-HOBA for 10 days. The present inventors found significant accumulation of isoLG protein adducts in esophageal animal tissues affected by reflux. The present inventors also found that treatment of animals with 2-HOBA prevents accumulation of isoLG adducts in vivo providing an interesting perspective on future applications of isoLG scavengers (FIG. 3A). Notably, the present inventors did not find significant difference between surgical animals treated with 2-HOBA and sham control animals, suggesting that 2-HOBA efficiently scavenged isoLGs in animal esophageal tissues (FIG. 3A).

To investigate further the isoLG adducts in vivo, 19 esophageal biopsies collected from GERD and normal patients were immunostained with D11 antibody and analyzed for isoLG adducts. The present inventors found that although levels of isoLG adducts were generally higher in GERD patients (n=10), individual patients showed high variability in isoLG accumulation; sixty percent of patients (6 out of 10 patients, staining score=3) showed strong staining, while other individuals did not show a significant difference from control group (FIG. 3B). These data may suggest that isoLG levels temporarily fluctuate in GERD patients following the dynamics of exposure of esophageal tissues to the refluxate. Interestingly, some GERD patients showed a strong nuclear staining showing that adducts form on nuclear proteins (FIG. 3B).

To investigate how induction of isoLG by reflux affects proteins in esophageal cells, the present inventors explored the regulation of p53 protein. Selection is based on an important role of p53 tumor suppressor in prevention of tumorigenic alterations in the esophagus. p53 regulates a number of critically important cellular processes, including apoptosis, cell cycle progression, cellular senescence, and repair from genotoxic damage. Not surprisingly, inhibition of p53 activity facilitates tumorigenesis. The present inventors have demonstrated that the treatment with acidic bile salts induce DNA damage but doesn't activate p53 in esophageal cells.

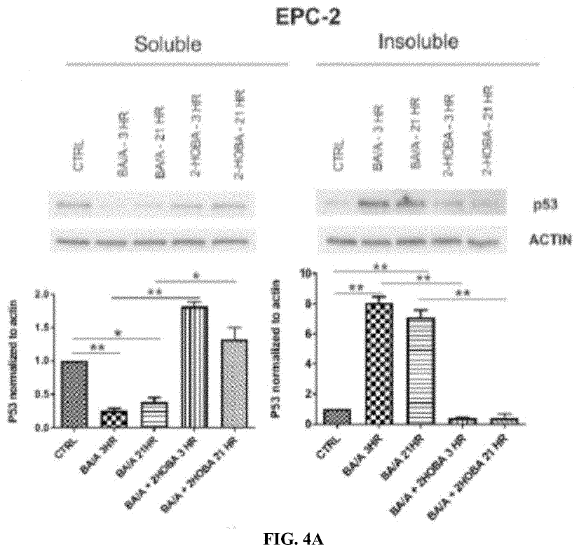

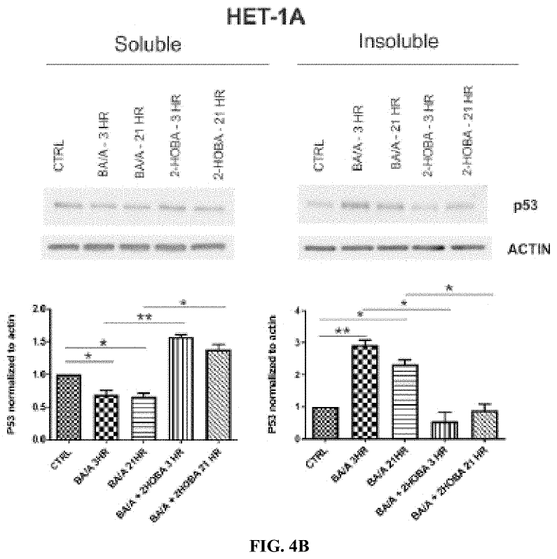

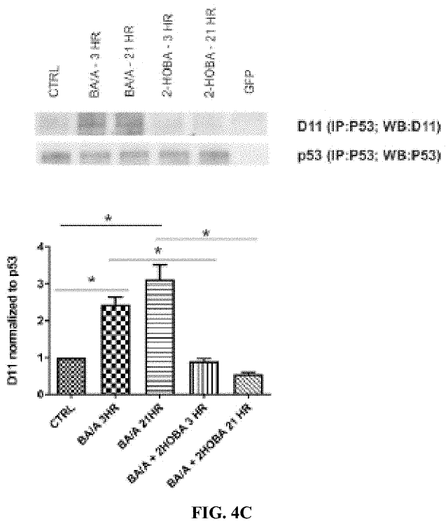

To investigate the regulation of p53 protein, non-transformed EPC-2 cells were treated with BA/A as described above and analyzed for expression of p53 by Western blotting. The present inventors separately analyzed soluble and insoluble cellular fractions, which were prepared as described in the Methods section. Unexpectedly, the present inventors found that levels of p53 protein were significantly decreased upon treatment with BA/A in soluble protein fraction, while insoluble protein fraction was enriched in precipitated p53 protein (FIG. 4A). A similar effect was also observed in another non-transformed cell line HET-1A (FIG. 4B). Given the strong hydrophobic properties of the isoLG adducts, it is likely that isoLG adducts affect the solubility of p53 protein. To test this hypothesis, the solubility of p53 was analyzed in the presence of isoLG scavenger 2HOBA. The present inventors found that treatment with 2-HOBA prevents precipitation of p53, suggesting that, at least in part, isoLG adducts are responsible for inactivation of p53 (FIGS. 4A and 4B). These findings are particularly interesting in the light of recent evidence that p53 protein forms insoluble protein aggregates in tumor cells that cause inactivation of p53.

To directly analyze p53 protein adducts, cellular lysates of EPC-2 cells, which were treated with BA/A in the presence or absence of 2-HOBA, were immunoprecipitated with p53 antibody. The immunoprecipitated p53 protein was then analyzed for the isoLG adducts by Western blotting with isoLG-specific D11 antibody. The present inventors found that BA/A lead to formation of p53 isoLG protein adducts. Importantly, 2-HOBA inhibited the formation of p53 protein adducts, further supporting our findings.

In summary, the present inventors revealed, for the first time, that gastroesophageal reflux leads to accumulation of isoLG protein adducts in the esophagus. The present inventors found isoLG protein adducts in animals, in which reflux was induced by surgical procedure, and in GERD patients. Our studies also revealed that exposure to acidic bile salts leads to the formation of p53 protein adducts causing precipitation and inhibition of p53. Importantly, The present inventors found that the isoLG scavenger 2HOBA efficiently suppresses accumulation of reflux-induced protein adducts in vitro and in vivo.

Methods

Cell Lines: Human non-tumorous esophageal cell line (HET-1A) and human esophageal carcinoma cell line (TE-7) were purchased from ATCC. The morphology, karyotyping and PCR based techniques were used by ATCC to confirm the cell line identity. HET-1A cells were cultured in DMEM (Life Technologies, Carlsbad, Calif.) and TE-7 in RPMI (Life Technologies, Carlsbad, Calif.) media. Both DMEM and RPMI were supplemented with 10% fetal bovine serum and 100 μg/ml penicillin/streptomycin. Human immortalized esophageal epithelial cells EPC-2 (kindly provided by Dr. Claudia Andl, University of Central Florida, Fla.) were cultured in keratinocyte SFM media supplemented with 40 gig/ml bovine pituitary extract, 1.0 ng/ml epidermal growth factor (Life Technologies, Carlsbad, Calif.), 5% fetal bovine serum and 100 μg/ml penicillin/streptomycin. All cells were cultured at 37° C. in an atmosphere of 5% CO2.

Treatment With Acidic Bile Salts (BA/A) and 2-HOBA: Cells were treated in acidic DMEM media (pH 4.0), containing bile salts cocktail at final concentration of 100 μM. The bile salt cocktail was prepared with a combination of glycocholic, taurocholic, glycodeoxycholic, glycochenodeoxycholic and deoxycholic sodium salts (all reagents were from Sigma-Aldrich, St. Louis, Mo.) at a concentration of 20 μM each. TE-7 and HET-1A cells were treated for 30 minutes, whereas, EPC-2 cells were treated for 15 minutes and then the media was replaced. IsoLG scavenger 2-hydroxybenzylamine (2HOBA) has been previously characterized. Final concentration of 2-HOBA was 50 μM.

Antibodies, Cell Fractionation and Immunoprecipitation: The following antibodies were used: β-actin (Sigma Aldrich, St. Louis, Mo.), p53 (DO-1) (Millipore, Burlington, Mass.) and anti-mouse IgG HRP (Promega, Madison, Wis.). D-11, an isoLG-lysyl adducts specific Scfv antibody, has been isolated from a phage display recombinant antibody library. The resulting Scfv antibody displays an E-tag recognized by an anti-E-tag antibodies (19). Anti-E tag HRP-conjugated secondary antibody from Abcam (Abcam, Cambridge, UK) was used.

After treatment with acidic bile salts, the cell lysates were sonicated and centrifuged at 16000 g at 4° C. for 20 minutes. The supernatant, which contains the soluble cellular fraction, was carefully collected. The remaining cell pellet, which contains the insoluble cellular fraction, was washed twice, resuspended in PBS containing protease inhibitor cocktail (Sigma Aldrich, St. Louis, Mo.) and sonicated. Both soluble and insoluble cellular fractions were analyzed by Western blotting as described previously.

For immunoprecipitation, cells were harvested by centrifugation at 200 g for 5 minutes and resuspended in PBS with protease inhibitor cocktail. Following sonication, the lysates were centrifuged twice at 16000 g at 4° C. for 20 min. The resulting supernatants were collected. p53 protein was immunoprecipitated with p53 (DO-1) and protein G agarose (Roche, Basel, Switzerland) with a ratio of 1 μg of antibodies per 1 mg of total protein. The immunoprecipitated samples were analyzed by Western blotting with D11 antibody and anti-E tag HRP-conjugated antibodies from Abcam. The analyzed membranes were stripped in Restore™ Western Blot Stripping Buffer (ThermoFisher Scientific, Waltham, Mass.) at +50° C. for 1 hour, washed, and blocked with 5% milk and analyzed by Western blotting with p53 (DO1) antibody.

Immunofluorescence: TE-7 and EPC-2 cells were grown on chamber slides, treated with BA/A for the indicated time and cultured in fresh media for 18 hours. Cells were then fixed in methanol:acetone at a 1:1 ratio (v/v). Non-specific binding was blocked with PBS-T (PBS with 0.1% Tween20) containing 1% BSA and 22 mg/ml glycine. Slides were incubated with D11 antibody for 16-18 h in a humidified chamber, followed by incubation with rabbit E-tag antibody for 1 hour and with goat anti-rabbit AlexaFluor 594 conjugated antibody (Invitrogen, Carlsbad, Calif.) for 1 hour. After washing with PBS, cells were counterstained with 4, 6-diamidino-2-phenylindole (DAPI) (ThermoFisher Scientific, Waltham, Mass.) for 1 minute and examined under a fluorescence microscope (Olympus, Pittsburgh, Pa.); at least 150 cells were assessed.

Induction of Reflux in Mice: Reflux was induced by surgical procedure, esophagojejunal anastomosis, as previously described. Esophagojejunostomy was performed on fourteen 8-week-old 129SV mice according to the protocol approved by the Vanderbilt University Animal Care and Use Committee. Animals were then be allowed a 2-weeks recovery period on regular rodent chow ad libitum prior to were randomly assigned into two groups. First group of 7 mice with surgery was treated with 2-HOBA (lmg/ml) delivered in drinking water for 10 days, while a control group (7 surgical animals) only received water. Additional control group of 5 animals with sham surgery received water. The esophagus and gastroesophageal junction areas from the test and control groups of animals were harvested, paraffin-embedded and analyzed by immunohistochemistry with D11 antibody. Indices of D11 positivity were scored and compared in test and control groups of animals.

Detection of isoLG Protein Adducts in GERD Patients: Nineteen archival esophageal biopsies collected at Vanderbilt University Medical Center from patients with GERD as well as patients without GERD symptoms were used for analyses. The use of all human pathology specimens for research was approved by the Institutional Review Board (IRB) of Vanderbilt University Medical Center. Since only de-identified tissues were included in this retrospective study, the IRB has waived the requirements for informed consent. Specimens were histologically verified and selected for immunohistochemical analyses. Immunohistochemical staining was done with Dl 1-E tag Antibody (1 μg/ml) and Anti-E tag HRP-conjugated antibody. The intensity of staining was graded as 0 (negative), 1 (weak), 2 (moderate) or 3 (strong). The frequency was graded according to the percentage of positive cells.

Detection of Levuglandinyl-Lysine Lactam Using Liquid Chromatography/Electrospray Ionization/Tandem Mass Spectrometry (LC/ESI/MS/MS): Levuglandinyl-lysine lactam was analyzed in TE-7 cells treated with acidic bile salts and control untreated cells using LC/ESI/MS/MS. The detailed protocol of analyses is included in Supplemental materials.

Statistics: Statistical analysis was performed using the Student t-test and Mann-Whitney tests, depending on the data set. Results were shown as mean±SEM. Results were considered significant if p<0.05.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

[1] Souza R F. From Reflux Esophagitis to Esophageal Adenocarcinoma. Dig Dis. 2016; 34(5):483-90.
[2] Napier K J, Scheerer M, and Misra S. Esophageal cancer: A Review of epidemiology, pathogenesis, staging workup and treatment modalities. World J Gastrointest Oncol. 2014; 6(5): 112-20.
[3] Bhardwaj V, Gokulan R C, Horvat A, Yermalitskaya L, Korolkova O, Washington K M, et al. Activation of NADPH oxidases leads to DNA damage in esophageal cells. Sci Rep. 2017; 7(1):9956.
[4] Jolly A J, Wild C P, and Hardie L J. Acid and bile salts induce DNA damage in human oesophageal cell lines. Mutagenesis. 2004; 19(4):319-24.
[5] Dulak A M, Stojanov P, Peng S, Lawrence M S, Fox C, Stewart C, et al. Exome and whole-genome sequencing of esophageal adenocarcinoma identifies recurrent driver events and mutational complexity. Nat Genet. 2013; 45(5):478-86.
[6] Qiao D, Gaitonde S V, Qi W, and Martinez J D. Deoxycholic acid suppresses p53 by stimulating proteasome-mediated p53 protein degradation. Carcinogenesis. 2001; 22(6):957-64.
[7] Roman S, Petre A, Thepot A, Hautefeuille A, Scoazec J Y, Mion F, et al. Downregulation of p63 upon exposure to bile salts and acid in normal and cancer esophageal cells in culture. Am J Physiol Gastrointest Liver Physiol. 2007; 293(1):G45-53.
[8] Zaika E, Wei J, Yin D, Andl C, Moll U, El-Rifai W, et al. p73 protein regulates DNA damage repair. FASEB J. 2011; 25(12):4406-14.
[9] Zhang H Y, Hormi-Carver K, Zhang X, Spechler S J, and Souza R F. In benign Barrett's epithelial cells, acid exposure generates reactive oxygen species that cause DNA double-strand breaks. Cancer Res. 2009; 69(23):9083-9.
[10] Davies S S, Amamath V, Montine K S, Bernoud-Hubac N, Boutaud O, Montine T J, et al. Effects of reactive gamma-ketoaldehydes formed by the isoprostane pathway (isoketals) and cyclooxygenase pathway (levuglandins) on proteasome function. FASEB J. 2002; 16(7):715-7.
[11] Mont S, Davies S S, Roberts Second L J, Mernaugh R L, McDonald W H, Segal B H, et al. Accumulation of isolevuglandin-modified protein in normal and fibrotic lung. Sci Rep. 2016; 6:24919.
[12] Kirabo A, Fontana V, de Faria A P, Loperena R, Galindo C L, Wu J, et al. DC isoketal-modified proteins activate T cells and promote hypertension. J Clin Invest. 2014; 124(10):4642-56.
[13] Dvorak K, Payne C M, Chavarria M, Ramsey L, Dvorakova B, Bernstein H, et al. Bile acids in combination with low pH induce oxidative stress and oxidative DNA damage: relevance to the pathogenesis of Barrett's oesophagus. Gut. 2007; 56(6):763-71.
[14] Boeckxstaens G E, and Rohof W O. Pathophysiology of gastroesophageal reflux disease. Gastroenterol Clin North Am. 2014; 43(1):15-25.
[15] Nehra D, Howell P, Williams C P, Pye J K, and Beynon J. Toxic bile acids in gastro-oesophageal reflux disease: influence of gastric acidity. Gut. 1999; 44(5):598-602.
[16] Yan H P, Roberts L J, Davies S S, Pohlmann P, Parl F F, Estes S, et al. Isolevuglandins as a gauge of lipid peroxidation in human tumors. Free Radic Biol Med. 2017; 106:62-8.
[17] Davies S S, Brantley E J, Voziyan P A, Amarnath V, Zagol-Ikapitte I, Boutaud O, et al. Pyridoxamine analogues scavenge lipid-derived gamma-ketoaldehydes and protect against H2O2-mediated cytotoxicity. Biochemistry. 2006; 45(51): 15756-67.
[18] Kim S, and An SS. Role of p53 isoforms and aggregations in cancer. Medicine (Baltimore). 2016; 95(26):e3993.
[19] Davies S S, Talati M, Wang X, Mernaugh R L, Amarnath V, Fessel J, et al. Localization of isoketal adducts in vivo using a single-chain antibody. Free Radic Biol Med. 2004; 36(9): 1163-74.
[20] Amarnath V, Amarnath K, Amarnath K, Davies S, and Roberts L J, 2nd. Pyridoxamine: an extremely potent scavenger of 1,4-dicarbonyls. Chem Res Toxicol. 2004; 17(3):410-5.
[21] Boutaud O, Li J, Zagol I, Shipp E A, Davies S S, Roberts L J, 2nd, et al. Levuglandinyl adducts of proteins are formed via a prostaglandin H2 synthase-dependent pathway after platelet activation. J Biol Chem. 2003; 278(19):16926-8.
[22] Boutaud O, Brame C J, Chaurand P, Li J, Rowlinson S W, Crews B C, et al. Characterization of the lysyl adducts of prostaglandin H-synthases that are derived from oxygenation of arachidonic acid. Biochemistry. 2001; 40(23):6948-55.

The invention thus being described, it would be understood that various details of the presently disclosed subject matter can be changed without departing from the scope of the subject matter disclosed herein. Furthermore, the foregoing description is for the purpose of illustration only, and not for the purpose of limitation.

We claim:

1. A method for suppressing or ameliorating accumulation of reflux-induced protein adducts to a subject in need thereof, comprising administering an effective amount of a compound of the following formula: 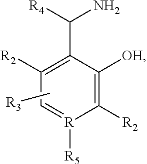
wherein:
R is C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N;
and stereoisomers and analogs thereof; and pharmaceutically acceptable salts thereof.

2. The method of claim 1, wherein the reflux-induced protein adducts comprise isolevyglandins (isoLG) adducts of proteins.

3. The method of claim 1, wherein the subject has GERD or is at risk of having GERD.

4. The method of claim 1, wherein the compound is 2-hydroxybenzylamine, methyl-2-hydroxybenzylamine, ethyl-2-hydroxybenzylamine.

5. The method of claim 1, wherein the compound is selected from the formula: 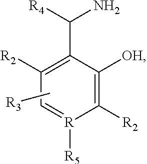
wherein:
R is CH, C—R₃, C—CH₃, or C—CH₂—CH₃;
R₂ is independently H, substituted or unsubstituted alkyl;
R₃ is independently H, halogen, alkoxy, hydroxyl, nitro;
R₄ is H, substituted or unsubstituted alkyl, carboxyl; and pharmaceutically acceptable salts thereof.

6. The method of claim 1, wherein the compound is 2-hydroxybenzylamine.

7. The method of claim 1, wherein the compound is chosen from:

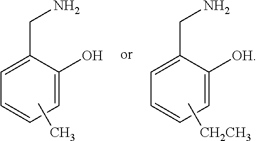

8. The method of claim 1, wherein the compound is chosen from: 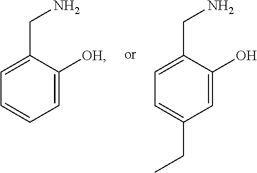
or a pharmaceutically acceptable salt thereof.

9. The method of claim 1, wherein the reflux-induced protein adducts comprise isolevyglandins (isoLG) adducts of proteins.

10. The method of claim 1, wherein the subject has GERD or is at risk of having GERD.

11. A method for treating, preventing, or ameliorating bile damage to the esophagus in a subject in need thereof, comprising administering to the subject a compound of the following formula: 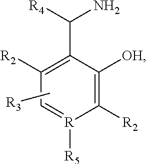
wherein:
R is C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

12. The method of claim 11, wherein the subject has GERD or is at risk of having GERD.

13. The method of claim 11, wherein the compound is 2-hydroxybenzylamine, methyl-2-hydroxybenzylamine, ethyl-2-hydroxybenzylamine.

14. The method of claim 11, wherein the compound is selected from the formula: 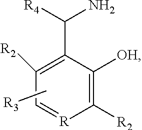
wherein:
R is CH, C—R₃, C—CH₃, or C—CH₂—CH₃;
R₂ is independently H, substituted or unsubstituted alkyl;
R₃ is independently H, halogen, alkoxy, hydroxyl, nitro;
R₄ is H, substituted or unsubstituted alkyl, carboxyl; and pharmaceutically acceptable salts thereof.

15. The method of claim 11, wherein the compound is 2-hydroxybenzylamine.

16. The method of claim 11, wherein the compound is chosen from:

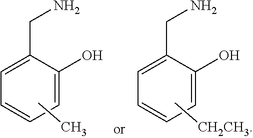

17. The method of claim 11, wherein the compound is be chosen from: 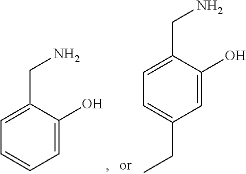
or a pharmaceutically acceptable salt thereof.

18. A method for ameliorating the risk of esophageal adenocarcinoma for a subject in need thereof, comprising administering to the subject a compound of the following formula: 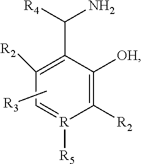
wherein:
R is C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

19. The method of claim 18, wherein the subject has GERD or is at risk of having GERD.

20. The method of claim 18, wherein the compound is 2-hydroxybenzylamine, methyl-2-hydroxybenzylamine, ethyl-2-hydroxybenzylamine.

21. The method of claim 18, wherein the compound is selected from the formula: 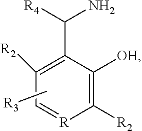
wherein:
R is CH, C—R₃, C—CH₃, or C—CH₂—CH₃;
R₂ is independently H, substituted or unsubstituted alkyl;
R₃ is independently H, halogen, alkoxy, hydroxyl, nitro;
R₄ is H, substituted or unsubstituted alkyl, carboxyl; and pharmaceutically acceptable salts thereof.

22. The method of claim 18, wherein the compound is 2-hydroxybenzylamine.

23. The method of claim 18, wherein the compound is chosen from:

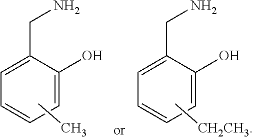

24. The method of claim 18, wherein the compound is be chosen from: 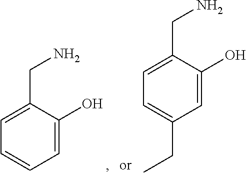
or a pharmaceutically acceptable salt thereof.

25. The method of claim 18, wherein the esophageal adenocarcinoma is reflux-induced.

26. A method of reducing or ameliorating reactive oxygen species produced by reflux for a subject in need thereof, comprising administering to a subject in need thereof a compound of the following formula: 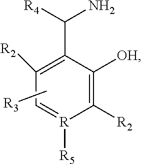
wherein:
R is C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; and pharmaceutically acceptable salts thereof.

27. The method of claim 26, wherein the subject has GERD or is at risk of having GERD.

28. The method of claim 26, wherein the compound is 2-hydroxybenzylamine, methyl-2-hydroxybenzylamine, ethyl-2-hydroxybenzylamine.

29. The method of claim 26, wherein the compound is selected from the formula: 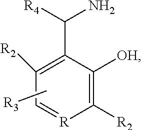
wherein:
R is CH, C—R₃, C—CH₃, or C—CH₂—CH₃;
R₂ is independently H, substituted or unsubstituted alkyl;
R₃ is independently H, halogen, alkoxy, hydroxyl, nitro;
R₄ is H, substituted or unsubstituted alkyl, carboxyl; and pharmaceutically acceptable salts thereof.

30. The method of claim 26, wherein the compound is 2-hydroxybenzylamine.

31. The method of claim 26, wherein the compound is chosen from:

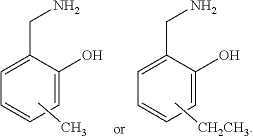

32. The method of claim 26, wherein the compound is be chosen from: 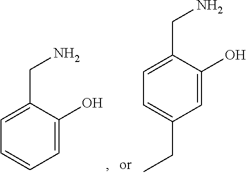
or a pharmaceutically acceptable salt thereof.

33. A method for treating, preventing, or ameliorating esophageal damage caused by reflux, comprising the step of co-administering to the subject at least one compound of the following formula: 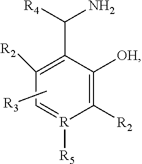
wherein:
R is C;
R₂ is independently H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₂, R₃ and R₄, and may cyclize with to one or more R₂, R₃, or R₅ to form an optionally substituted C₃₋₈ membered ring containing C, O, S or N;
R₃ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂ or R₅ to form an optionally substituted C₃₋₈ membered ring containing C, O, S or N;
R₄ is H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₅ to form an optionally substituted C₃₋₈ membered ring containing C, O, S or N;
R₅ is a bond, H, hydroxy, halogen, nitro, CF₃, C₁₋₆ alkyl, C₁₋₆ alkoxy, C₃₋₁₀ cycloalkyl, C₃₋₈ membered ring containing C, O, S or N, optionally substituted with one or more R₄, R₂ and R₃ may cyclize with to one or more R₂, R₃, or R₄ to form an optionally substituted C₃₋₈ membered ring containing C, O, S or N; and stereoisomers and analogs thereof; with a drug having a known side effect of treating, preventing, or ameliorating reflux.

34. The method of claim 33, wherein the subject has GERD or is at risk of having GERD.

35. The method of claim 33, wherein the compound is 2-hydroxybenzylamine, methyl-2-hydroxybenzylamine, ethyl-2-hydroxybenzylamine.

36. The method of claim 33, wherein the compound is selected from the formula: 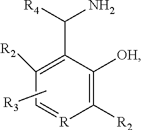
wherein:
R is CH, C—R₃, C—CH₃, or C—CH₂—CH₃;
R₂ is independently H, substituted or unsubstituted alkyl;
R₃ is independently H, halogen, alkoxy, hydroxyl, nitro;
R₄ is H, substituted or unsubstituted alkyl, carboxyl; and pharmaceutically acceptable salts thereof.

37. The method of claim 33, wherein the compound is 2-hydroxybenzylamine.

38. The method of claim 33, wherein the compound is chosen from:

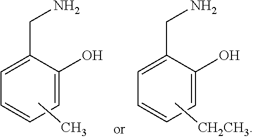

39. The method of claim 33, wherein the compound is chosen from: 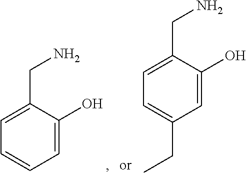
or a pharmaceutically acceptable salt thereof.
